(19) United States
(12) Patent Application Publication
(10) Pub. No.: US 2014/0213482 A1
(43) Pub. Date: Jul. 31, 2014

(54) SIMPLE METHOD FOR DETCTING PLURIPOTENT STEM CELLS GENETICALLY MODIFIED BY HOMOLOGOUS RECOMBINATION

(71) Applicant: Kyoto University, Kyoto (JP)

(72) Inventors: Shinya Yamanaka, Kyoto-shi (JP); Kenji Osafune, Kyoto-shi (JP)

(21) Appl. No.: 14/345,756

(22) PCT Filed: Sep. 20, 2012
(86) PCT No.: PCT/JP2012/074076

§ 371 Date: Mar. 19, 2014

(30) Foreign Application Priority Data
Sep. 20, 2011 (JP) 2011-204950

(51) Int. Cl. C12N 15/85

(52) U.S. Cl. CPC C12N 15/85
USPC 506/9

(57) ABSTRACT

The present invention relates to a method for producing pluripotent stem cells, which comprises the steps of introducing an artificial chromosome having a genetically modified chromosome fragment as a targeting vector, and determining the number of some or all copies of the introduced artificial chromosome using an SNP array, so as to select pluripotent stem cells modified by homologous recombination, and, a method for detecting pluripotent stem cells genetically modified by homologous recombination.

### TECHNICAL FIELD

The present invention relates to a method for detecting pluripotent stem cells genetically modified by homologous recombination, and, a method for producing pluripotent stem cells genetically modified by homologous recombination, which is characterized by comprising the detection method.

### BACKGROUND ART

Gene targeting that involves artificially modifying a desired site of the endogenous genomic DNA of an organism (Patent Literature 1 and Patent Literature 2) is performed as a method for imparting biological properties that are not innate properties of an organism to the organism, suppressing the expression of biological properties that are innate properties of an organism, and analyzing the functions of a gene of the organism by partially modifying (deletion, insertion or substitution) the endogenous genomic DNA.

Gene targeting is used for knocking out an endogenous gene (Non-Patent Literature 1 and Non-Patent Literature 2) or knocking in an exogenous sequence into a chromosome. However, this method requires much efforts since the efficiency thereof is very low (10⁻⁶ to 10⁻⁹ cells among transfected cells).

Accordingly, gene targeting has been refined to involve cleaving a desired site using meganuclease, in order to increase the gene targeting efficiency 1,000-fold or more (Non-Patent Literature 3, Non-Patent Literature 4, Non-Patent Literature 5, Non-Patent Literature 6, Non-Patent Literature 7, and Non-Patent Literature 8).

However, such a technique still requires a confirmation step in order to determine whether or not clones have been modified by homologous recombination. Hence, a method for efficiently detecting clones that have been modified as desired from among numerous candidates is required.

### PRIOR ART LITERATURE

### Patent Literature

Patent Literature 1: WO1990/11354
Patent Literature 2: WO1991/09955

### Non-Patent Literature

Non-Patent Literature 1: Capecchi, M. R., Science, (1989) 244: 1288-1292
Non-Patent Literature 2: Smithies, O., Nature Medicine, (2001) 7: 1083-1086
Non-Patent Literature 3: Puchta H, et al., Nucleic Acids Res., (1993) 21: 5034-5040
Non-Patent Literature 4: Choulika A, et al., Mol. Cell. Biol., (1995) 15: 1968-1973
Non-Patent Literature 5: Puchta H, et al., Proc. Natl. Acad. Sci. U.S.A., (1996) 93: 5055-5060
Non-Patent Literature 6: Sargent R G, et al., Mol. Cell. Biol., (1997) 17: 267-277
Non-Patent Literature 7: Cohen-Tannoudji M, et al., Mol. Cell. Biol., (1998) 18: 1444-1448
Non-Patent Literature 8: Donoho G, et al., Mol. Cell. Biol., (1998) 18: 4070-4078

### SUMMARY OF THE INVENTION

### Problem to Be Solved by the Invention

One objective of the present invention is to provide a method that comprises detecting whether or not homologous recombination has taken place at a desired site or region on the genome upon preparation of pluripotent stem cells genetically modified by homologous recombination.

Specifically, the object of the present invention is to detect pluripotent stem cells wherein a desired locus has been modified by gene targeting. More specifically, the object of the present invention is to provide a method for distinguishing pluripotent stem cells wherein homologous recombination has taken place at a desired site on the genome from pluripotent stem cells wherein random integration has taken place.

### Means for Solving the Problem

To achieve the above objectives, the present inventors have focused on a method for detecting copy number polymorphism using an SNP (single nucleotide polymorphism) array method, and thus have discovered that whether or not homologous recombination has taken place can be determined by determining via the SNP array method the number of copies of a region corresponding to a chromosome fragment in pluripotent stem cells into which an artificial chromosome vector having the genetically modified chromosome fragment has been introduced as a targeting vector. Furthermore, a method using an incorporated selection marker as an index and/or a method for determining the number of alleles having a wild-type sequence at a modification site are combined in order to obtain secondary verification of whether or not homologous recombination has taken place. Thus the present inventors have completed the present invention.

Specifically, the present invention is as described below.
[1] A method for producing pluripotent stem cells genetically modified by homologous recombination, comprising the following steps (1) to (3) of:
(1) introducing an artificial chromosome having a genetically modified chromosome fragment into pluripotent stem cells, so as to prepare a population consisting of pluripotent stem cell clones assumed to be genetically modified;
(2) determining the number of some or all copies of the introduced artificial chromosome using an SNP array for the above population of pluripotent stem cell clones; and
(3) selecting pluripotent stem cell clones in which the above number of copies is equivalent to or lower than the same number of wild-type cells into which no artificial chromosomes have been introduced, as pluripotent stem cells genetically modified by homologous recombination.
[2] The method according to [1], wherein the genetic modification of the step (1) comprises incorporation by which an exogenous DNA fragment is inserted into a cellular genome while the endogenous sequence is retained.
[3] The method according to [2], wherein the exogenous DNA is a DNA encoding a selection marker, and the method further comprises a step of selecting clones that are positive for the selection marker.
[4] The method according to any one of [1] to [3], further comprising a step of selecting pluripotent stem cell clones in which the number of alleles subjected to genetic modification is lower than the same number of wild-type cells not modified by homologous recombination.
[5] The method according to any one of [1] to [4], wherein the above pluripotent stem cells are human pluripotent stem cells.
[6] The method according to any one of [1] to [5], wherein the above artificial chromosome is a BAC clone.
[7] The method according to [3], wherein the above selection marker is a drug resistance marker.
[8] A method for detecting pluripotent stem cells genetically modified by homologous recombination, comprising a step of determining, when pluripotent stem cells genetically modified by homologous recombination are produced, the number of copies of a recombined region using an SNP array, and then detecting pluripotent stem cell clones in which the number of copies is equivalent to or lower than the same number of wild-type cells not modified by homologous recombination, as pluripotent stem cells genetically modified by homologous recombination.
[9] The method according to [8], wherein the genetic modification is incorporation by which an exogenous DNA fragment is inserted into the cellular genome while the endogenous sequence is retained.
[10] The method according to [9], wherein the exogenous DNA is a DNA encoding a selection marker, and the method further comprises a step of detecting clones positive for the selection marker, as pluripotent stem cells genetically modified by homologous recombination.
[11] The method according to any one of [8] to [10], further comprising a step of detecting pluripotent stem cell clones in which the number of alleles subjected to genetic modification is lower than the same number of wild-type cells not modified by homologous recombination, as pluripotent stem cells genetically modified by homologous recombination.
[12] The method according to any one of [8] to [11], wherein the pluripotent stem cells are human pluripotent stem cells.
[13] The method according to [10], wherein the selection marker is a drug resistance marker.

This description includes all or part of the contents as disclosed in the description and/or drawings of Japanese Patent Application No. 2011-204950 (filing date: Sep. 20, 2011), from which the present application claims the priority.

### Effect of the Invention

Through the use of the present invention, genetic modification of pluripotent stem cells by homologous recombination can be conveniently detected by efficient procedures. The method of the present invention can be combined with a highly efficient gene targeting technique, so that the method of the present invention contributes to improvement in the overall efficiency of genetic modification of pluripotent stem cells.

### BRIEF DESCRIPTION OF THE DRAWINGS

FIG. 1 shows a scheme for inserting a GFP-PGK-Neo cassette into the human OSR1 (odd-skipped related 1; M. Katoh, Int. J. Mol. Med. 2002; 10(2): 221-225) locus using a Cre-loxP system.

FIG. 2(A) shows a graph obtained by quantitating the abundance of wild-type regions containing the OSR1 initiation codon in the chromosome of a parent line or that of a drug-resistant clone prepared by introduction of a modified BAC clone. FIG. 2(B) shows the result of analyzing the number of copies of each probe in the vicinity of the OSR1 locus of human chromosome 2 of each iPS cell line (3D36, 3D45, 3F3, 3149, or 3D12) using an SNP array.

### MODES FOR CARRYING OUT THE INVENTION

The present invention will be described below in detail. The present invention provides a method for producing pluripotent stem cells genetically modified by homologous recombination, which is characterized by comprising the following steps (1) to (3) of:
(1) introducing an artificial chromosome having a genetically modified chromosome fragment into pluripotent stem cells, so as to prepare a population consisting of pluripotent stem cell clones assumed to be genetically modified;
(2) determining the number of some or all copies of the introduced artificial chromosome using an SNP array for the above population of pluripotent stem cell clones;
(3) selecting pluripotent stem cell clones in which the above number of copies is equivalent to or lower than the same number of wild-type cells into which no artificial chromosomes have been introduced, as pluripotent stem cells genetically modified by homologous recombination.

The term “homologous recombination” as used herein refers to a gene targeting means for artificially modifying a specific gene on a chromosome or a genome. When a genomic fragment having a portion homologous to that of a target sequence on the chromosome is introduced into cells, the term refers to recombination that takes place based on the nucleotide sequence homology between the introduced genomic fragment and the locus corresponding thereto on the chromosome.

Also, the term “genetic modification” refers to, in the locus of a desired gene on the chromosome, the insertion of an exogenous DNA, the substitution of a portion of or the whole of the gene with an exogenous DNA, or the deletion of the gene. More specifically, genetic modification refers to the insertion (that is, “knock-in”) of an exogenous DNA fragment while the endogenous DNA sequence is retained in a manner such that the fragment is expressed in conjunction with the expression of a gene at a specific locus or is expressed constitutively, or, the substitution, deletion, or disruption (that is, “knock-out”) of a portion of or the whole gene sequence so as to modify the endogenous DNA sequence. Moreover, when a target pluripotent stem cell has a mutation in a specific gene, the term “genetic modification” for performing a recombination by which the gene is substituted with a normal gene sequence refers to the modification of the gene to be a normal gene.

A gene to be subjected to modification may be adequately selected depending on the purpose. Examples thereof include, but are not limited to, a causative gene of a disease, a marker gene serving as an index of a cell type, and a housekeeping gene, the expression level of which is not decreased. Examples of cell types include endodermal cells, ectodermal cells, mesodermal cells, chordamesodermal cells, paraxial mesodermal cells, intermediate mesodermal cells, lateral plate mesodermal cells, nerve cells, glial cells, hematopoietic cells, hepatocytes, pancreatic p cells, renal precursor cells, endothelial cells, pericytes, epithelial cells, osteoblasts, myoblasts, and chondrocytes. Examples of marker genes serving as indices for these cell types include, but are not limited to, GATA4, GATA5, GATA6, AFP, HNF-3β, SOX17, FOXA2, PDGFRα, FLK1, Brahcyury, Gremlin, MYH2, Nestin, SOX13, SOX21, CryM, Otx2, TP63, SOX2, PSA-NCAM, TuJ1, Thy1.2, GFAP, PAX6, A2B5, CD11b, c-kit, CD34, CD90, CD117, Albumin, CK18, CK19, PDX1, OSR1, SIX2, GATA2, VEGFR2, NG2, desmin, MUC1, BGLAP, SPP1, MyoD, MYF5, Myogenin, Aggrecan, Collagen II, and Sox9. These gene sequences are available from known DNA databases including the NCBI GenBank (hhtp://www.ncbi.nlm.nih.gov), EMBL, and DDBJ.

### <Method for Introducing Artificial Chromosome>

In the present invention, the term “artificial chromosome” refers an artificially prepared chromosome having functions required for replication in host cells, such as a replication origin, centromere, and telomere. Examples thereof include an Escherichia coli-derived BAC vector (Shizuya et al., (1992) Proc. Natl. Acad. Sci. U.S.A. 89: 8794-8797), a P1 phage-derived PAC vector (Ioannou et al., (1994) Nature Genetics 6: 84-89; Pierce et al., (1992) Meth. Enzymol. 216: 549-574; Pierce et al. (1992) Proc. Natl. Acad. Sci. U.S.A., 89: 2056-2060; U.S. Pat. No. 5,300,431 and International PCT Application No. WO92/14819), a yeast-derived YAC vector (Burke et al., (1987) Science 236: 806-812), a human-derived HAC vector (WO1998/008964), and a mammal-derived MAC vector (JP Patent Publication (Kokai) No. 2000-517182 A, JP Patent Publication (Kokai) No. 2007-306928 A). These artificial chromosomes can be proliferated within host cells while a huge-size genomic fragment including an exogenous DNA is retained. Such an artificial chromosome is preferably an artificial chromosome having a chromosome fragment and is more preferably an artificial chromosome having a human chromosome fragment. The size of a DNA fragment is a size that enables stable replication of the artificial chromosome within the host. In the case of BAC or PAC, the size is generally about 300 kb or less. In the case of YAC, the size is 1 Mb or less. In the case of HAC and MAC, the size can be 1 Mb or more.

In the method of the present invention, any one of the above examples of artificial chromosomes can be used depending on the size of a genomic fragment to be introduced. In a preferred embodiment, an artificial chromosome is a BAC vector (H. Shizuya et al., Proc. Natl. Acad. Sci. U.S.A., 1992; 89: 8794-8797; U J Kim et al., Genomics 1996; 34:213-218; S. Asakawa et al., Gene 1997; 191: 69-79; M R Green and J Sambrook, Molecular Cloning A Laboratory Manual Fourth Edition, 2012, Chapter 5, Cold Spring Harbor Laboratory Press). More specifically, an example of a BAC vector containing a human chromosome fragment is RP-11 that is the library of 437,000 clones having human genomic fragments with an average of 175 kb prepared by the Roswell Park Cancer Institute, U.S.A. Such a BAC vector having a genomic fragment contained in the library is referred to as “BAC clone.”

An artificial chromosome having a chromosome fragment can be prepared using a method known by persons skilled in the art. Examples of such a method include a method that involves cleaving a chromosome fragment at a desired position therein using a restriction enzyme, and then ligating a DNA fragment having an adequate functional sequence thereto, and a method that involves performing homologous recombination within Escherichia coli using a phage-derived Red gene. A kit having a plasmid that expresses the Red gene can be purchased from Gene Bridges, with which an artificial chromosome can be modified according to the protocols included therewith. Preparation and application of BAC, PAC and YAC artificial chromosomes are described in, for example, M R Green and J Sambrook, Molecular Cloning A Laboratory Manual Fourth Edition, 2012, Chapter 5, Cold Spring Harbor Laboratory Press. Furthermore, a MAC or HAC artificial chromosome is a vector containing a centromere, telomeres, and (long arm and short arm) chromatin portions that are induced from a single or a plurality of chromosomes of a mammal such as a human. This vector can be constructed using a technique such as telomere truncation. A foreign gene or locus can be inserted into a chromatin portion (JP Patent Publication (Kokai) No. 2011-177145 A, JP Patent Republication (Saikohyo) No 2008-013067, WO2004/031385).

Examples of an exogenous DNA to be inserted into an artificial chromosome for genetic modification include, but are not particularly limited to, useful (human or non-human animal-derived) genes and selection marker genes, or combinations thereof. A cassette for incorporation of an exogenous DNA may further contain a promoter, IRES, a recognition sequence for site-specific recombinase, a terminator, and the like.

An exogenous DNA is preferably a DNA having useful biological functions, medical functions, or useful functions for selection of clones.

Examples of the term “useful (human or non-human animal-derived) genes” as used herein include, but are not limited to, genes useful for medical research or useful at a practical level, such as genes with unknown functions, the causative genes of diseases, and genes useful for treatment, marker genes serving as cell-type indices, and housekeeping genes the expression levels of which are not decreased.

The term “selection marker gene” as used herein refers to a gene that functions as an index for selection of a host cell. As selection markers, either known positive markers or negative markers can be used. Examples of positive selection markers include, but are not limited to, fluorescent markers, light-emitting markers, and drug resistance markers. Examples of “fluorescent markers” include, but are not limited to, genes encoding fluorescent proteins such as a green fluorescent protein (GFP), a cyan (blue) fluorescent protein (CFP), a yellow fluorescent protein (YFP), and a red fluorescent protein (dsRed). Examples of “light-emitting markers” include, but are not limited to, genes encoding luminescent proteins such as luciferase. Examples of “drug resistance markers” include, but are not limited to, a fusion gene (β-geo gene) with a neomycin (G418) resistance gene, a CAT gene, a GFP gene, an SV40 large T gene, a neomycin resistance gene, a puromycin resistance gene, a hygromycin resistance gene, and a blasticidin S resistance gene.

Moreover, examples of the “site-specific recombinase” include Cre recombinase (Gorman C, Bullock C. Curr Opin Biotechnol. (2000), 11: 455-60), and FLP recombinase (Buchholz F, et al., Nat Biotechnol. (1998), 16: 657-662). Examples of target sequences for these examples of recombinase include loxP and FRT. For the purpose of deleting a region between two recognition sequences, it is desired to insert the recognition sequences into two positions between which a desired region is located.

A terminator is a polyadenylation signal as a transcription termination sequence. Examples of a polyadenylation signal sequence include, but are not particularly limited to, human BGH poly A, SV40 poly A, human p actin poly A, rabbit β globulin poly A, and immunoglobulin κ poly A.

The above exogenous DNA may be appropriately placed in an artificial chromosome depending on the purpose such as insertion, substitution, or deletion, so that it can function. For example, for the purpose of substitution and deletion of genes to be modified, a selection marker and a terminator may be ligated to and placed within the gene sequence following the initiation codon of the target gene. Alternatively, a selection marker may be ligated to an endogenous promoter and placed, so that it is expressed in conjunction with the expression of a gene to be modified. For this purpose, a selection marker may also be ligated and placed together with the 2A self-cleaving peptide of foot-and-mouth disease virus (see Science, 322, 949-953, 2008, for example), an IRES sequence, and the like. Furthermore, a selection marker to be inserted may be ligated in advance to an exogenous promoter for constant expression thereof, in order to confirm that the artificial chromosome has been introduced into cells. Examples of a promoter to be used herein include a PGK promoter, an EF-α promoter, a CAG promoter, an SRα promoter, an SV40 promoter, an LTR promoter, a CMV (cytomegalovirus) promoter, an RSV (Rous sarcoma virus) promoter, MoMuLV (Moloney murine leukemia virus) LTR, and an HSV-TK (herpes simplex virus thymidine kinase) promoter.

Examples of methods for introducing an artificial chromosome into cells include a calcium phosphate precipitation method (Graham et al., (1978) Virology 52: 456-457, Wigler et al., (1979) Proc. Natl. Acad. Sci. U.S.A. 76 1373-1376 and Current Protocols in Molecular Biology Vol.1, Wiley Inter-Science, Supplement 14, Unit 9.1.1-9.1.9 (1990)), a fusion method using polyethylene glycol (U.S. Pat. No. 4,684,611), a method using lipid carriers such as lipofection (Teifel et al., (1995) Biotechniques 19: 79-80, Albrecht et al., (1996) Ann. Hematol. 72: 73-79; Holmen et al., (1995) In Vitro Cell Dev. Biol. Anim. 31: 347-351, Remy et al., (1994) Bioconjug. Chem. 5: 647-654, Le Bolc'het al., (1995) Tetrahedron Lett. 36: 6681-6684, Loeffler et al., (1993) Meth. Enzymol, 217: 599-618 and Strauss (1996) Meth. Mol. Biol. 54: 307-327), electroporation, and methods for fusion with microcells (U.S. Pat. Nos. 5,240,840, 4,806,476, 5,298,429, and 5,396,767, Fournier (1981) Proc. Natl. Acad. Sci. U.S.A. 78: 6349-6353 and Lambert et al., (1991) Proc. Natl. Acad. Sci. U.S.A. 88: 5907-59).

A population consisting of pluripotent stem cell clones assumed to be genetically modified is prepared by the above techniques. Here, the expression “ . . . assumed to be genetically modified” means that most pluripotent stem cell clones have been genetically modified, but some genetically unmodified (that is, wild-type) clones can be mixed therein. Such clones include not only clones resulting from genetic modification at desired sites or regions on the genome (that is, homologous recombination), but also clones resulting from random genetic modification (that is, randomly integrated) on the genome. In the present invention, clones genetically modified by homologous recombination are selected from such a population consisting of pluripotent stem cell clones assumed to be genetically modified, using an SNP array described as follows. A method for determining the number of copies using an SNP array has never been applied to the detection of pluripotent stem cells modified by homologous recombination. In the present invention, target cell clones modified by homologous recombination can be easily recognized using such a technique.

### <SNP Array>

In the present invention, the term “SNP (single nucleotide polymorphism) array” refers to an array for SNP typing using an allele-specific oligonucleotide probe, which is preferably capable of detecting the number of genomic copies using the quantitative properties of the array signals. Such an SNP array to be preferably used herein has oligonucleotide probes for determination of copy number polymorphism at average probe intervals ranging from 2.5 kb to 7 kb. Examples thereof include Affymetrix SNP array 6.0, illumina CNV370-Duo and Bead Chips.

In the method of the present invention, for determination of the number of copies using an SNP array, chromosomal DNA is not directly used as a sample, but may be used after amplification of the extracted chromosomal DNA. An example of a method for amplification involves treating with a restriction enzyme such as Hind III, Xba I, Nsp I or Sty I, adding an adaptor to the 5′ end and the 3′ end, and then amplifying by PCR using adaptor-specific primers. Regarding the amplification, selective amplification of only short restriction enzyme fragments with a length between 0.5 kb and 1.5 kb is desired. Furthermore, a group of DNA fragments prepared by more finely fragmenting the thus amplified DNA fragment with DNase I is hybridized to an array having an oligonucleotide probe, and then the amount of the fragments can be detected based on the signal intensity. The obtained signal intensity data can be calculated as the number of copies using CNAG (copy number analyzer for gene chip) software (http://www.genome.umin.jp/).

For detection of more precise number of copies, it is desirable to detect signals in an allele-specific manner. For this purpose, a pluripotent stem cell that has not undergone homologous recombination or another clone obtained by introduction of an artificial chromosome is used as a control and the signal intensity thereof is measured similarly using an SNP array and then analyzed using AsCNAR (allele-specific copy-number analysis using anonymous references) (http://www.genome.umin.jp/), so that the signal intensity can be obtained for each allele. In this manner, a problem such that precise number of copies cannot be calculated when alleles differ in their affinity for the same probe can be solved by measuring signals in an allele-specific manner.

An array that can be used in the present invention is preferably provided in the form of microarray onto which an oligonucleotide serving as a probe is immobilized on a solid-phase support (substrate). Examples of a solid-phase support of a microarray include a glass substrate, a silicon substrate, a membrane, and beads. The material, size, and shape thereof are not particularly limited. A method for forming a microarray is not particularly limited and any method that can be used by persons skilled in the art may be used. An example thereof is a method (on-chip method) that involves directly synthesizing a probe on a solid-phase support surface, or a method that involves binding a probe prepared in advance to a solid-phase support surface. When a probe is directly synthesized on a solid-phase support surface, a method that is generally employed involves performing selective synthesis of an oligonucleotide within a predetermined fine matrix region through the combination of photolithography technology that is used for semiconductor production and solid phase synthesis technology using a protecting group that is selectively removed by photoirradiation. Meanwhile, examples of a method that can be used for binding a probe prepared in advance to a solid-phase support surface include a method that involves spotting a probe onto the surface of a solid-phase support that has been surface-treated with a polycation compound, a silane coupling agent or the like having an amino group, an aldehyde group, an epoxy group or the like using a spotter device depending on the type of probe nucleic acid or solid-phase support, and a method that involves synthesizing a probe by introducing a reaction-active group, spotting the probe onto a solid-phase support surface that has been surface-treated to form a reactive group in advance, and thus binding and immobilizing the probe to the solid-phase support surface via covalent bonding.

The number of copies of a chromosome fragment in a chromosome (genome) obtained from pluripotent stem cells into which the artificial chromosome having the genetically modified chromosome fragment has been introduced is determined, so that the number of copies of the chromosome fragment contained in the artificial chromosome in the cellular genome can be confirmed. At this time, in the case of homologous recombination with the chromosome of pluripotent stem cells, the resulting number of copies of the chromosome fragment in the pluripotent stem cells is equivalent to (that is, the same as) the number of copies confirmed before the introduction of the artificial chromosome, or lower than the number of copies confirmed before the introduction of the artificial chromosome. When an exogenous DNA fragment is inserted (that is, knocked-in) into a cellular genome while the endogenous DNA sequence is retained, the resulting number of copies is equivalent to the same number confirmed before the above introduction. When the whole or a portion of the gene sequence is substituted, deleted, or disrupted (that is, knock out) so as to modify the endogenous DNA sequence, the resulting number of copies is lower than the same number confirmed before the above introduction. Meanwhile, when a chromosome fragment is randomly incorporated (that is, random integration) into a cellular genome, the resulting number of copies is higher than the same number confirmed before the above introduction, such as 3 copies or more. Therefore, through determination of the number of copies by the specified method, the presence or the absence of homologous recombination, specifically knock-in type or knock-out type homologous recombination can be determined.

### <Supplementary Selection Using Selection Marker>

In the present invention, the presence or the absence of homologous recombination can be determined by determining the number of copies using an SNP array as described above. In this case, a selection marker can also be used supplementarily.

Specifically, in the present invention, a selection marker is used after introduction of an artificial chromosome having the selection marker into cells, so that cells into which the artificial chromosome has been incorporated into the chromosome (or the genome) can be selected. When a drug selection marker is used herein, the corresponding drug is added to the cell culture solution, so that cells into which an artificial chromosome has been incorporated into the chromosome (or the genome) via homologous recombination or random integration can be selectively obtained as cells confirmed positive for the selection marker.

### <Supplementary Selection by Determination of the Number of Alleles>

Furthermore, in the present invention, a supplementary selection method that involves determining the number of alleles can be performed. According to this method, when one allele having a wild-type sequence that has not been genetically modified can be confirmed, or, an allele having a wild-type sequence cannot be confirmed, in a chromosome (or a genome) of pluripotent stem cells after the introduction of the artificial chromosome having a genetically modified chromosome fragment into the pluripotent stem cells, the cells can be selected as clones genetically modified by homologous recombination. Preferably, pluripotent stem cell clones, in which the number of genetically modified alleles is lower than, and preferably ½ the number of alleles of wild-type cells not modified by homologous recombination, are detected as pluripotent stem cells genetically modified by homologous recombination. A wild-type gene on the autosome generally comprises 2 alleles. However, when one of the genes is modified, the wild-type gene comprises 1 allele. Therefore, when the resulting number of genetically modified alleles is a half of the same number of wild-type cells, the presence of modification at the target locus can be confirmed. An example of a method for determining the number of alleles having a wild-type sequence is a method that involves amplifying by a PCR method a region containing a site at which a gene has been modified based on chromosomal DNA extracted from pluripotent stem cells subjected to recombination, measuring the amount of the PCR product of a size characteristic of the wild-type sequence (when the wild-type sequence is contained), and comparing with pluripotent stem cells (as wild-type cells) that have not been genetically modified. Another embodiment is a method that involves cleaving a chromosome extracted from genetically modified pluripotent stem cells with an arbitrary restriction enzyme, and then measuring DNA fragments of the specific size resulting from gene modification by the Southern blot method.

Pluripotent stem cells that can be genetically modified by the method of the present invention are as specifically described below.

### <Pluripotent Stem Cells>

Pluripotent stem cells are stem cells having both pluripotency, by which the cells are capable of differentiating into all cells existing in an organism, and, proliferation potency. Examples of these pluripotent stem cells include, but are not particularly limited to, embryonic stem (ES) cells, embryonic stem (nt ES) cells from clone embryos obtained by nuclear transplantation, Germline stem cells (“GS cells”), embryonic germ cells (“EG cells”), induced pluripotent stem (iPS) cells, and cultured fibroblasts- or bone marrow stem cell-derived pluripotent cells (Muse cells). Examples of preferable pluripotent stem cells include ES cells, nt ES cells, and iPS cells.

### (A) Embryonic Stem Cells

ES cells are stem cells having pluripotency and proliferation potency via self-replication, which are established from inner cell mass of early embryos (e.g., blastocysts) of a mammal such as a human or a mouse.

ES cells are stem cells from embryos originated from inner cell mass of blastocysts that are embryos after the 8-cell stage of fertilized eggs and the morula stage. ES cells have so-called pluripotency, by which they are capable of differentiating into all cells composing an adult, and proliferation potency via self-replication. ES cells were discovered in mice in 1981 (M. J. Evans and M. H. Kaufman (1981), Nature 292: 154-156). Thereafter, ES cell lines were established in primates including humans, monkeys, and the like (J. A. Thomson et al. (1998), Science 282:1145-1147; J. A. Thomson et al. (1995), Proc. Natl. Acad. Sci. U.S.A., 92:7844-7848; J. A. Thomson et al. (1996), Biol. Reprod., 55:254-259; J. A. Thomson and V. S. Marshall (1998), Curr. Top. Dev. Biol., 38:133-165).

ES cells can be established by removing inner cell mass from blastocysts of fertilized eggs of a subject animal and then culturing the inner cell mass on fibroblasts as feeders. Also, cell maintenance by subculture can be carried out using a culture solution supplemented with substances such as a leukemia inhibitory factor (LIF) and a basic fibroblast growth factor (bFGF). Methods for establishment and maintenance of human and monkey ES cells are described in U.S. Pat. No. 5,843,780; Thomson J A, et al. (1995), Proc Natl. Acad. Sci. U.S.A., 92: 7844-7848; Thomson J A, et al., (1998), Science. 282: 1145-1147; H. Suemori et al. (2006), Biochem. Biophys. Res. Commun., 345:926-932; M. Ueno et al. (2006), Proc. Natl. Acad. Sci. U.S.A., 103:9554-9559 ; H. Suemori et al. (2001), Dev. Dyn., 222:273-279; H. Kawasaki et al. (2002), Proc. Natl. Acad. Sci. U.S.A., 99: 1580-1585; and Klimanskaya I, et al. (2006), Nature. 444: 481-485, for example.

As a culture solution for preparation of ES cells, a DMEM/F-12 culture solution supplemented with 0.1 mM 2-mercaptoethanol, 0.1 mM nonessential amino acid, 2 mM L-glutamic acid, 20% KSR, and 4 ng/ml b-FGF is used, for example. Human ES cells can be maintained under wet atmosphere of 2% CO₂/98% air at 37° C. (O. Fumitaka et al. (2008), Nat. Biotechnol., 26: 215-224). Also, it is necessary for ES cells to subculture every 3 to 4 days. At this time, subculture can be carried out using 0.25% trypsin and 0.1 mg/ml collagenase IV in PBS containing 1 mM CaCl₂ and 20% KSR, for example.

ES cells can be generally selected by Real-Time PCR using the expression of a gene marker such as alkaline phosphatase, Oct-3/4 or Nanog as an index. In particular, for selection of human ES cells, the expression of a gene marker such as OCT-3/4, NANOG, or ECAD can be used as an index (E. Kroon et al. (2008), Nat. Biotechnol., 26: 443-452).

Human ES cell lines such as WA01(H1) and WA09(H9) are available from the WiCell Research Institute, and KhES-1, KhES-2, and KhES-3 are available from the Institute for Frontier Medical Sciences, Kyoto University (Kyoto, Japan).

### (B) Germline Stem Cells

Germline stem cells are testis-derived pluripotent stem cells, serving as an origin for spermatogenesis. Germline stem cells can also be induced to differentiate into cells of various lines in a manner similar to that of ES cells. For example, the cells have properties such that a chimeric mouse can be produced when transplanted into mouse blastocysts (M. Kanatsu-Shinohara et al. (2003) Biol. Reprod., 69: 612-616; K. Shinohara et al. (2004), Cell, 119: 1001-1012). Germline stem cells are self-replicable in a culture solution containing a glial cell line-derived neurotrophic factor (GDNF) and Germline stem cells can be obtained by repeated subculture of the cells under culture conditions similar to those for ES cells (Masanori Takebayashi et al., (2008), Experimental Medicine, Vol. 26, No. 5 (Extra Number), pp. 41-46, YODOSHA (Tokyo, Japan)).

### (C) Embryonic Germ Cells

Embryonic germ cells are cells established from primordial germ cells at the prenatal period and have pluripotency similar to that of ES cells. Embryonic germ cells can be established by culturing primordial germ cells in the presence of substances such as LIF, bFGF, and a stem cell factor (Y. Matsui et al. (1992), Cell, 70: 841-847; J. L. Resnick et al. (1992), Nature, 359: 550-551).

### (D) Induced Pluripotent Stem Cells

Induced (artificial) pluripotent stem (iPS) cells can be prepared by introducing a specific reprogramming factor in the form of DNA or protein into somatic cells. iPS cells are somatic cell-derived artificial stem cells having properties almost equivalent to those of ES cells, such as pluripotency and proliferation potency via self-replication (K. Takahashi and S. Yamanaka (2006) Cell, 126: 663-676; K. Takahashi et al. (2007) Cell, 131: 861-872; J. Yu et al. (2007) Science, 318: 1917-1920; Nakagawa M et al., (2008) Nat. Biotechnol., 26: 101-106 (2008); International Publication WO 2007/069666). Reprogramming factors may be composed of a gene, a gene product thereof or non-coding RNA specifically expressed in ES cells, a gene, a gene product thereof or non-coding RNA playing an important role in maintenance of undifferentiation of ES cells, or a low molecular weight compound. Examples of genes contained in such reprogramming factors include Oct3/4, Sox2, Sox1, Sox3, Sox15, Sox17, K1f4, K1f2, c-Myc, N-Myc, L-Myc, Nanog, Lin28, Fbx15, ERas, ECAT15-2, Tc11, beta-catenin, Lin28b, Sa111, Sa114, Esrrb, Nr5a2, Tbx3 and Glis1. These reprogramming factors may be used independently or in combination. Examples of a combination of reprogramming factors include those described in WO2007/069666, WO2008/118820, WO2009/007852, WO2009/032194, WO2009/058413, WO2009/057831, WO2009/075119, WO2009/079007, WO2009/091659, WO2009/101084, WO2009/101407, WO2009/102983, WO2009/114949, WO2009/117439, WO2009/126250, WO2009/126251, WO2009/126655, WO2009/157593, WO2010/009015, WO2010/033906, WO2010/033920, WO2010/042800, WO2010/050626, WO 2010/056831, WO2010/068955, WO2010/098419, WO2010/102267, WO2010/111409, WO2010/111422, WO2010/115050, WO2010/124290, WO2010/147395, WO2010/147612, Huangfu D, et al. (2008), Nat. Biotechnol., 26: 795-797, Shi Y, et al. (2008), Cell Stem Cell, 2: 525-528, Eminli S, et al. (2008), Stem Cells. 26:2467-2474, Huangfu D, et al. (2008), Nat Biotechnol. 26:1269-1275, Shi Y, et al. (2008), Cell Stem Cell, 3, 568-574, Zhao Y, et al. (2008), Cell Stem Cell, 3:475-479, Marson A, (2008), Cell Stem Cell, 3, 132-135, Feng B, et al. (2009), Nat Cell Biol. 11:197-203, R. L. Judson et al., (2009), Nat. Biotech., 27:459-461, Lyssiotis C A, et al. (2009), Proc Natl Acad Sci U.S.A. 106:8912-8917, Kim J B, et al. (2009), Nature. 461:649-643, Ichida J K, et al. (2009), Cell Stem Cell. 5:491-503, Heng J C, et al. (2010), Cell Stem Cell. 6:167-74, Han J, et al. (2010), Nature. 463:1096-100, Mali P, et al. (2010), Stem Cells. 28:713-720, and Maekawa M, et al. (2011), Nature. 474: 225-9.

Examples of the above reprogramming factors include histone deacetylase (HDAC) inhibitors [e.g., low-molecular-weight inhibitors such as valproic acid (VPA), trichostatin A, sodium butyrate, MC 1293, and M344, and nucleic acid expression inhibitors such as siRNA and shRNA against HDAC (e.g., HDAC1 siRNA Smartpool™ (Millipore) and HuSH 29mer shRNA Constructs against HDAC1 (OriGene))], MEK inhibitors (e.g., PD184352, PD98059, U0126, SL327, and PD0325901), Glycogen synthase kinase-3 inhibitors (e.g., Bio and CHIR99021), DNA methyltransferase inhibitors (e.g., 5′-azacytidine), histone methyltransferase inhibitors (e.g., low-molecular-weight inhibitors such as BIX-01294, and nucleic acid expression inhibitors such as siRNA and shRNA against Suv39h1, Suv39h2, SetDB1 and G9a), L-channel calcium agonists (e.g., Bayk8644), butyric acid, TGFβ inhibitors or ALK5 inhibitors (e.g., LY364947, SB431542, 616453, and A-83-01), p53 inhibitors (e.g., siRNA and shRNA against p53), ARID3A inhibitors (e.g., siRNA and shRNA against ARID3A), miRNA such as miR-291-3p, miR-294, miR-295, and mir-302, Wnt Signaling (e.g., soluble Wnt3a), neuropeptide Y, prostaglandins (e.g., prostaglandin E2 and prostaglandin J2), and factors to be used for enhancing the efficiency of establishment, such as hTERT, SV40LT, UTF1, IRX6, GLIS1, PITX2, and DMRTB1. In the Description, these factors used for improving the efficiency of establishment are not particularly distinguished from the reprogramming factors.

Reprogramming factors in the form of protein may be introduced into somatic cells by a technique such as lipofection, fusion with a cell membrane-permeable peptide (e.g., HIV-derived TAT and polyarginine), or microinjection.

Meanwhile, reprogramming factors in the form of DNA can be introduced into somatic cells by a technique such as a technique using a vector such as a virus, a plasmid, or an artificial chromosome, lipofection, a technique using a liposome, or microinjection. Examples of a viral vector include a retrovirus vector, a lentivirus vector (these are according to Cell, 126, pp. 663-676, 2006; Cell, 131, pp. 861-872, 2007; and Science, 318, pp. 1917-1920, 2007), an adenovirus vector (Science, 322, 945-949, 2008), an adeno-associated virus vector, and a Sendai virus vector (WO 2010/008054). Also, examples of an artificial chromosome vector include a human artificial chromosome (HAC), a yeast artificial chromosome (YAC), and a bacterial or phage artificial chromosome (BAC and PAC). As a plasmid, a plasmid for mammalian cells can be used (Science, 322: 949-953, 2008). A vector can contain regulatory sequences such as a promoter, an enhancer, a ribosome binding sequence, a terminator, and a polyadenylation site, so that a nuclear reprogramming substance can be expressed. A vector may further contain as necessary a drug resistance gene (e.g., a kanamycin resistance gene, an ampicillin resistance gene, and a puromycin resistance gene), a selection marker sequence such as a thymidine kinase gene and a diphtheria toxin gene, and a reporter gene sequence such as a green fluorescent protein (GFP), β glucuronidase (GUS), and FLAG. Furthermore, the above vector may contain LoxP sequences flanking a gene encoding a reprogramming factor, or, a promoter and a gene encoding a reprogramming factor binding to the promoter, so as to excise the gene or both the gene and the promoter after introduction of the vector into somatic cells.

Moreover, reprogramming factors in the form of RNA may be introduced into somatic cells by techniques such as lipofection or microinjection. For suppression of degradation, RNA prepared to incorporate 5-tnethyleytidine and pseudouridine (TriLink Biotechnologies) may also be used (Warren L, (2010) Cell Stem Cell. 7:618-630).

Examples of a culture solution for inducing iPS cells include a DMEM, DMEM/F12, or DME culture solution containing 10-15% FBS (these culture solutions may further appropriately contain LIF, penicillin/streptomycin, puromycin, L-glutamine, nonessential amino acids, β-mercaptoethanol, and the like), commercially available culture solutions [e.g., a culture solution for mouse ES cell culture (TX-WES culture solution (Thromb-X)), and a culture solution for primate ES cell culture (a culture solution for primate ES/iPS cells, ReproCELL, Kyoto, Japan), and serum-free media (mTeSR, Stemcell Technology)].

An example of culture methods is as follows. Somatic cells are brought into contact with reprogramming factors on a DMEM or DMEM/F12 culture solution containing 10% FBS at 37° C. in the presence of 5% CO₂ and are cultured for about 4 to 7 days. Subsequently, the cells are reseeded on feeder cells (e.g., mitomycin C-treated STO cells or SNL cells). About 10 days after contact between the somatic cells and the reprogramming factors, cells are cultured in a bFGF-containing culture solution for primate ES cell culture. About 30-45 days or more after the contact, iPS cell-like colonies can be formed.

Alternatively, cells may be cultured at 37° C. in the presence of 5% CO₂ using a DMEM culture solution containing 10% FBS (which may further appropriately contain LIF, penicillin/streptomycin, puromycin, L-glutamine, nonessential amino acids, β-mercaptoethanol, and the like) on feeder cells (e.g., mitomycin C-treated STO cells or SNL cells). After about 25-30 days or more, ES cell-like colonies can be formed. A desirable example of a method involves the direct use of somatic cells to be reprogrammed, instead of feeder cells (Takahashi K, et al. (2009), PLoS One. 4: e8067 or WO2010/137746), or extracellular matrix (e.g., Laminin-5 (WO2009/123349) and Matrigel (BD)).

Another example is a method that involves culturing with the use of a serum-free medium (Sun N, et al. (2009), Proc Natl Acad Sci U.S.A. 106: 15720-15725). Furthermore, for enhancement of the efficiency of establishment, iPS cells may be established under low-oxygen conditions (oxygen concentration of 0.1% or more and 15% or less) (Yoshida Y, et al. (2009), Cell Stem Cell. 5:237-241 or WO2010/013845).

During the above culture, a culture solution is exchanged with a fresh culture solution once a day from day 2 after the start of culture. In addition, the number of somatic cells to be used for nuclear reprogramming is not limited, but ranges from about 5×10³ to about 5×10⁶ cells per culture dish (100 cm²).

iPS cells can be selected based on the shape of the thus formed colonies. On the other hand, when a drug resistance gene that is expressed in conjunction with a gene (e.g., Oct3/4, Nanog) that is expressed when somatic cells are reprogrammed is introduced as a marker gene, established iPS cells can be selected by culturing in a culture solution (selective culture solution) containing the corresponding drug. Moreover, iPS cells can be selected by observation under a fluorescence microscope when the marker gene is a fluorescent protein gene, by adding a luminescent substrate when the marker gene is a luminescent enzyme gene, or by adding a chromogenic substrate when the marker gene is a chromogenic enzyme gene.

The term “somatic cells” as used herein may refer to all animal cells (preferably, mammalian cells including human cells) other than germ-line cells (e.g., ova and oocytes) or totipotent cells, or ES cells. Examples of somatic cells include, but are not limited to, fetal somatic cells, neonatal somatic cells, and mature healthy or pathogenic somatic cells. Examples thereof further include primary cultured cells, passage cells, and established cell lines. Specific examples of somatic cells include (1) tissue stem cells (somatic stem cells) such as neural stem cells, hematopoietic stem cells, mesenchymal stem cells, and dental pulp stem cells, (2) tissue precursor cells, (3) differentiated cells such as lymphocytes, epithelial cells, endothelial cells, muscle cells, fibroblasts (e.g., skin cells), hair cells, hepatocytes, gastric mucosal cells, enterocytes, splenocytes, pancreatic cells (e.g., pancreatic exocrine cells), brain cells, pneumocytes, renal cells and fat cells.

When iPS cells are used as materials for cells for transplantation, somatic cells with the HLA genotype that is the same or substantially the same as that of an organism to which the cells are transplanted are desirably used to avoid rejection. Here, the term “substantially the same” means that the HLA genotypes agree to such an extent that immunoreaction to transplanted cells can be suppressed by an immunosuppressive agent. An example is a somatic cell with the HLA type with 3 loci (HLA-A, HLA-B and HLA-DR) or 4 loci (HLA-C in addition to HLA-A, HLA-B and HLA-DR) that agree with the 3 or 4 loci of the organism.

### (E) Clone Embryo-Derived ES Cells Obtained by Nuclear Transplantation

nt ES cells are clone embryo-derived ES cells prepared by a nuclear transplantation technique, having almost the same properties as those of fertilized egg-derived ES cells (T. Wakayama et al., (2001), Science, 292: 740-743; S. Wakayama et al., (2005), Biol. Reprod., 72: 932-936; J. Byrne et al. (2007), Nature, 450:497-502). Specifically, nt ES (nuclear transfer ES) cells are established from inner cell mass of blastocysts from a clone embryo that has been obtained by substitution of the nucleus of an unfertilized egg with the nucleus of a somatic cell. For preparation of nt ES cells, the nuclear transplantation technique (J. B. Cibelli et al. (1998), Nature Biotechnol., 16: 642-646) and the ES cell preparation technique (above) are used in combination (Sayaka Wakayama et al., (2008), Experimental Medicine, Vol. 26, No. 5 (extra number), pp. 47-52). In nuclear transplantation, reprogramming can be performed by injecting the nucleus of a somatic cell into an unfertilized mammalian egg that has been enucleated, and then culturing the resultant for several hours.

### (F) Multilineage-Differentiating Stress Enduring Cells (Muse Cells)

Muse cells are pluripotent stem cells produced by the method described in WO2011/007900. Specifically, muse cells are pluripotent cells obtained by treating fibroblasts or bone marrow stromal cells with trypsin for a long time and preferably for 8 or 16 hours, and then performing suspension culture. Muse cells are positive for SSEA-3 and CD105.

### EXAMPLES

Examples and comparative examples of the present invention are as described below. These examples present an embodiment to assist the reproduction of the present invention, but do not limit the scope of the present invention.

### <Preparation of Recombined Human BAC Clone>

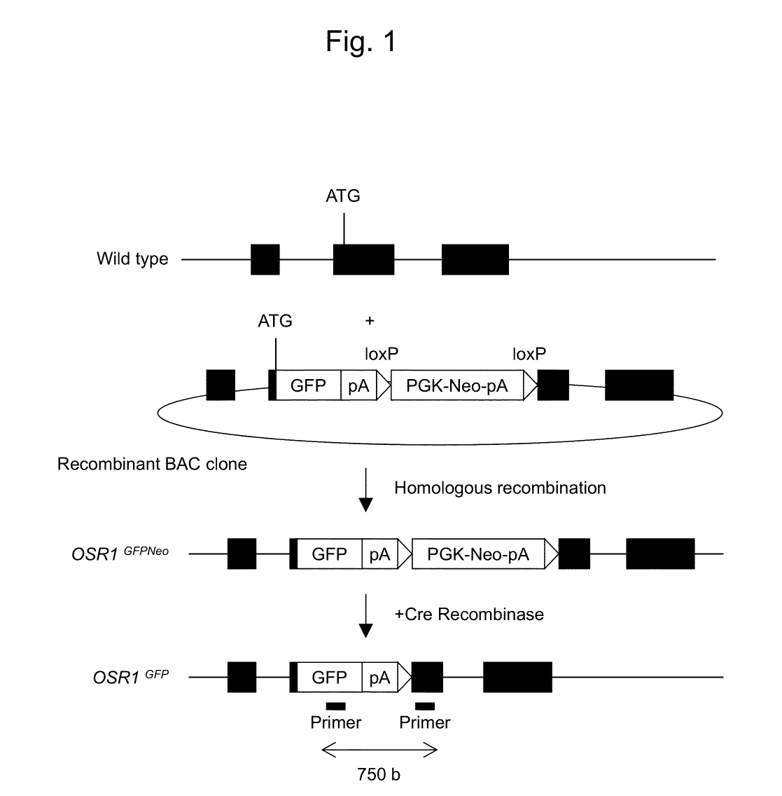

A human BAC clone (RP11-458J18) containing a region that extends 86.3 kb upstream and 89.8 kb downstream from the OSR1 locus was purchased from the BACPAC Resources Center (Oakland, Calif.). The clone was recombined by the method described in Lee, E. C. et al., (2001) Genomics 73, 56-65. Briefly, an EGFP-polyA-LoxP-PGK-Neo-LoxP (EGFP-pA-PNL) cassette having homology arms on the 5′ side and the 3′ side was prepared by PCR using hOSR1-EGFP-S: TCTTCTTTTCTTTGCAGATCCGGATTGAGAAGCCACTGCAACTACC GAACACCATGGTGAGCAAGGGCGAGGA (SEQ ID NO: 1) and hOSR1-PNL-AS: GTTCACTGCCTGAAGGAAGGAGTAGTTGGTGAGCTGCAGGGAAGG GTGGAGTCGACGGCGAGCTCAGACG (SEQ ID NO: 2) primers. This cassette and human BAC clone were introduced into Escherichia coli DH10B. Recombinase was activated for homologous recombination. The EGFP-pA-PNL cassette was inserted to immediately follow the OSR1 initiation codon in the human BAC clone (recombinant BAC clone in FIG. 1).

### <Preparation of iPS Cells Modified by Homologous Recombination>

The recombinant human BAC clone prepared by the above method was cleaved with a restriction enzyme, thereby preparing a single-stranded DNA. 30 μg of the single-stranded DNA was introduced into human iPS cells (201B7) treated with Y27632 (Wako (Tokyo, Japan); (R)-(+)-trans-N-(4-pyridyl)-4-(1-aminoethyl)-cyclohexanecarboxamide 2HCl.H₂O; ROCK (Rho-associated coiled-coil forming kinase) inhibitor) and trypsin by electroporation (250 V, 500 mF, single pulse). Two hours after introduction, iPS cells were cultured in a medium for drug selection.

### <PCR Analysis>

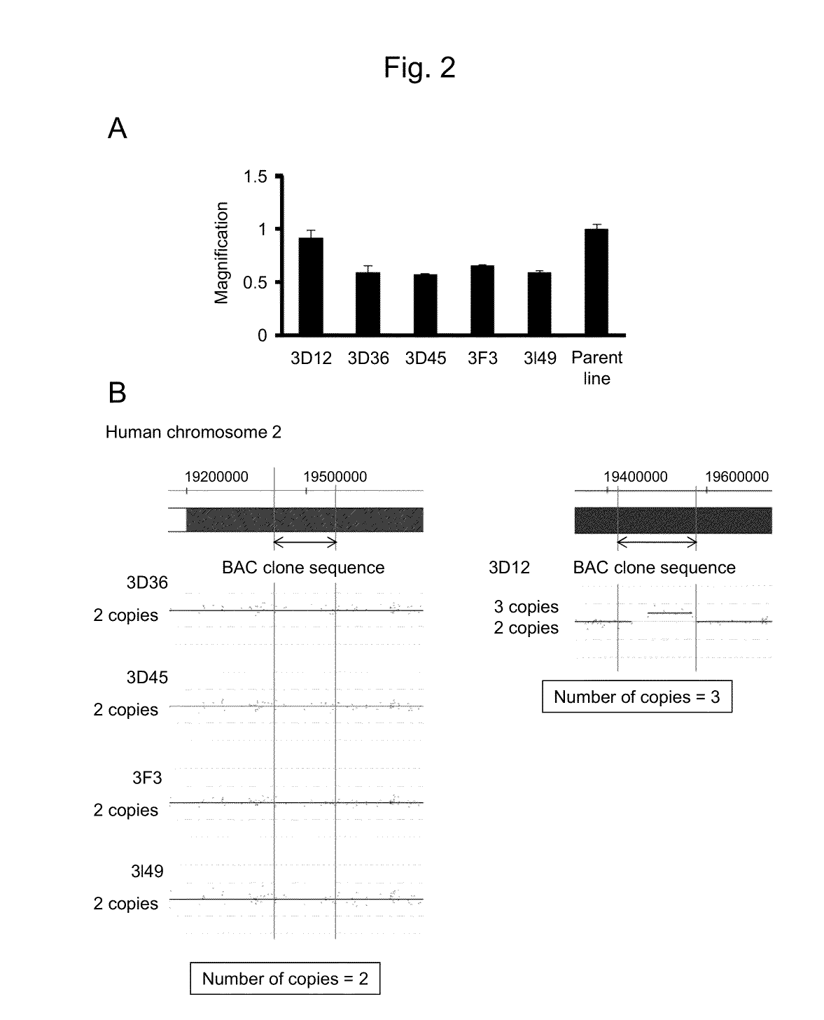

Chromosomal DNA was extracted from the thus obtained 130 drug-resistant iPS cell clones, and then subjected to quantitative PCR using OSR1F (5′-GGATTGAGAAGCCACTGCAACT-3′ (SEQ ID NO: 3)) and OSR1R (5′-CCGTTCACTGCCTGAAGGA-3′ (SEQ ID NO: 4)) primers (FIG. 2A). As a result, 4 clones (3D36, 3D45, 3F3 and 3149) were confirmed to be deficient in the regions neighboring the OSR1 initiation codon.

### <SNP Array Analysis>

Chromosomal DNA was extracted, and then data were obtained using GeneChip™ Mapping 250K NSP arrays (Affymetrix). Allele-specific copy number analysis was conducted using software (CNAG/AsCNAR) for 3D36, 3D45, 3F3, 3149 and 3D12. The number of copies of probe regions obtained from the detected value of each probe contained in GeneChip™ determined by CNAG/AsCNAR analysis was plotted on the vertical axis and the positions of the probes on the gene were plotted on the horizontal axis (FIG. 2B). As a result, 3D36, 3D45, 3F3 and 3149 were each found to contain 2 copies of the OSR1 locus region. This suggests the insertion of the EGFP-pA-PNL cassette by homologous recombination. Meanwhile, 3D12 was found to contain 3 copies of the OSR1 region. This suggests that the recombinant human BAC clone was incorporated into the chromosome not by homologous recombination, but by random incorporation. Therefore, human iPS cells (3D36, 3D45, 3F3 and 3149) wherein the EGFP-pA-PNL cassette had been knocked into desired positions by homologous recombination were obtained. The region to be analyzed was expanded and examined. As a result, 3149 was confirmed to have copy number polymorphism in chromosome 9. Accordingly, it was considered that, in the case of 3149, abnormal gene duplication had taken place in chromosome 9 during culture or genetic modification.

### <Karyotype Analysis>

Karyotype analysis was conducted by G band analysis, and 3D45 was confirmed to have normal karyotype.

<Treatment with Cre Recombinase>

Human iPS cells (3D36, 3D45, 3F3 and 3149) in which OSR1 had been targeted were cultured in media supplemented with 10 μM Y27632 for 1 day, and then subjected to separation by trypsin treatment. A Cre expression vector was introduced by electroporation. After introduction, chromosomal DNA was subjected to PCR using the primers shown in FIG. 1 that had been designed to follow the GFP sequence site and the initiation codon of the OSR1 gene, so as to include loxP sequences therebetween. Thus, the elimination of PGK-Neo-pA was confirmed for all clones.

### INDUSTRIAL APPLICABILITY

According to the present invention, pluripotent stem cells in which homologous recombination has taken place can be selectively produced in a highly efficient manner with the use of an artificial chromosome as a targeting vector. Moreover, the use of an SNP array makes it possible to conveniently detect pluripotent stem cells modified by homologous recombination. Therefore, pluripotent stem cells modified by homologous recombination are produced using the present invention, making it possible to conveniently screen for a therapeutic agent or an inducer of differentiation or to secure cells for treatment.

All publications, patents, and patent applications cited herein are incorporated herein by reference in their entirety.

1. A method for producing pluripotent stem cells genetically modified by homologous recombination, comprising the following steps (1) to (3) of:
(1) introducing an artificial chromosome having a genetically modified chromosome fragment into pluripotent stem cells, so as to prepare a population consisting of pluripotent stem cell clones assumed to be genetically modified;
(2) determining the number of some or all copies of the introduced artificial chromosome using an SNP array for the population of pluripotent stem cell clones; and
(3) selecting pluripotent stem cell clones in which the number of copies is equivalent to or lower than the same number of wild-type cells into which no artificial chromosomes have been introduced, as pluripotent stem cells genetically modified by homologous recombination.

2. The method according to claim 1, wherein the genetic modification of the step (1) comprises incorporation by which an exogenous DNA fragment is inserted into a cellular genome while the endogenous sequence is retained.

3. The method according to claim 2, wherein the exogenous DNA is a DNA encoding a selection marker, and the method further comprises a step of selecting clones that are positive for the selection marker.

4. The method according to any one of claims 1 to 3, further comprising a step of selecting pluripotent stem cell clones in which the number of alleles subjected to genetic modification is lower than the same number of wild-type cells not modified by homologous recombination.

5. The method according to any one of claims 1 to 4, wherein the pluripotent stem cells are human pluripotent stem cells.

6. The method according to any one of claims 1 to 5, wherein the artificial chromosome is a BAC clone.

7. The method according to claim 3, wherein the selection marker is a drug resistance marker.

8. A method for detecting pluripotent stem cells genetically modified by homologous recombination, comprising a step of determining, when pluripotent stem cells genetically modified by homologous recombination are produced, the number of copies of a recombined region using an SNP array, and then detecting pluripotent stem cell clones in which the number of copies is equivalent to or lower than the same number of wild-type cells not modified by homologous recombination, as pluripotent stem cells genetically modified by homologous recombination.

9. The method according to claim 8, wherein the genetic modification is incorporation by which an exogenous DNA fragment is inserted into the cellular genome while the endogenous sequence is retained.

10. The method according to claim 9, wherein the exogenous DNA is a DNA encoding a selection marker, and the method further comprises a step of detecting clones positive for the selection marker, as pluripotent stem cells genetically modified by homologous recombination.

11. The method according to any one of claims 8 to 10, further comprising a step of detecting pluripotent stem cell clones in which the number of alleles subjected to genetic modification is lower than the same number of wild-type cells not modified by homologous recombination, as pluripotent stem cells genetically modified by homologous recombination.

12. The method according to any one of claims 8 to 11, wherein the pluripotent stem cells are human pluripotent stem cells.

13. The method according to claim 10, wherein the selection marker is a drug resistance marker.


SEQUENCE LISTING (4 sequences)

SEQ ID NO: 1 - DNA, 73 bp, Artificial
  primer
tcttcttttctttgcagatccggattgagaagccactgcaactaccgaacaccatggtgagcaagggcgagga

SEQ ID NO: 2 - DNA, 70 bp, Artificial
  primer
gttcactgcctgaaggaaggagtagttggtgagctgcagggaagggtggagtcgacggcgagctcagacg

SEQ ID NO: 3 - DNA, 22 bp, Artificial
  primer
ggattgagaagccactgcaact

SEQ ID NO: 4 - DNA, 19 bp, Artificial
  primer
ccgttcactgcctgaagga